(19) United States
(12) Patent Application Publication
(10) Pub. No.: US 2002/0177834 A1
(43) Pub. Date: Nov. 28, 2002

(54) Absorbent article with dynamic air flow

(75) Inventors: James Arthur Davis, Roswell, GA (US); Wanda Walton Jackson, Alpharetta, GA (US); Jark C. Lau, Roswell, GA (US); K. Y. Wang, Alpharetta, GA (US); Susan Carol Paul, Alpharetta, GA (US); Rebecca Willey Griffin, Woodstock, GA (US); Roland Columbus Smith JR., Gainsville, GA (US); David Craige Strack, Canton, GA (US); Eugenio Go Varona, Marietta, GA (US); Marlene Rebecca Waugaman, Alpharetta, GA (US); Tamara Lee Mace, Dorville, GA (US)

Correspondence Address: William W. Letson, Kimberly-Clark Worldwide, Inc., Patent Department, 401 North Lake Street, Neenah, WI, 54956, US

(73) Assignee: Kimberly-Clark Worldwide, Inc.

(21) Appl. No.: 09/866,326

(22) Filed: May 25, 2001

(51) Int. Cl. A61F 13/15

(52) U.S. Cl. 604/385.12

(57) ABSTRACT

The present invention relates to an absorbent article comprising a topsheet, a backsheet, and a valving means, the valving means comprising at least one one-way valve and a connection means which permits the connection of a pumping means, wherein the valving means allows air to be distributed into one or more regions of the article. The valving means of the article allows air to be discharged into the article in a region between a wearer's skin and the product.

### FIELD OF THE INVENTION

The present invention relates to an air moving structure in an article for personal care like diapers, training pants, swimwear, absorbent underpants, adult incontinence products, wound dressings, bandages, feminine hygiene products, and absorbent pads or the like.

### BACKGROUND OF THE INVENTION

Absorbent articles include such items as personal care products, and more specifically diapers, training pants, swimwear, wound dressings, bandages, incontinence garments, absorbent pads and feminine hygiene products such as sanitary napkins, panty-liners and tampons and the like. The most basic design of all such articles typically includes a bodyside liner, an outercover and an absorbent core disposed between the bodyside liner and the outercover. Generally, the bodyside liner and the outercover are sealed about the periphery so as to encapsulate the absorbent core and thus make it possible to entrap and retain any fluids contained within the absorbent core. Depending upon the design of the particular personal care absorbent article, other components also may be included. Thus, the product may include such things as elastic side panels, fluid containment flaps, fastening devices and other layers of fluid transfer or retention materials.

The lack of air flow into personal care products has been a long standing concern because of adverse skin effects which are believed to be promoted by continuous exposure to moisture and the enzymes and other substances in bodily fluids. Many attempts to correct these adverse effects have been made, including the provision of materials designed to wick and hold liquids away from the skin, or to increase the breathability or air permeability of the personal care product components. Breathable diaper outercovers, for example, have been developed to allow increased air exchange and flow into the product and so improve the environment adjacent the skin. The condition of the wearer's skin is thus an area of continuing concern to the personal care product industry.

In light of low air flow within absorbent articles, more specifically personal care products, even those with breathable outercovers, it is an object of this invention to provide a dynamically breathable personal care product wherein air may be forced into or extracted from the product in the region between the skin and the product. Dynamic breathability, i.e., the forcing of air into a product, should not be confused with the provision of breathable materials which merely allow air to pass through them in a passive fashion.

It is a further object of this invention to provide a dynamically breathable absorbent article having a valving means comprising at least one valve and a connection means which permits the connection of a pumping means, wherein the valving means allows air to be distributed into or drawn from one or more regions of the article. It is a further object of the present invention that a pumping means can be attached or fixed to the absorbent article or external thereto. It is also an object of the present invention to utilize a valving means or pumping means containing a desiccant so as to further reduce the relative humidity of the air being forced into or extracted from the absorbent article.

### SUMMARY OF THE INVENTION

The present invention relates to an absorbent article comprising a topsheet, a backsheet, and a valving means, the valving means comprising at least one one-way valve and a connection means which permits the connection of a pumping means, wherein the valving means allows air to be distributed into one or more regions of the article. The valving means of the article allows air to be discharged into the article in a region between a wearer's skin and the product. In one embodiment, the present invention may further include a pump or pumping means, the pump or pumping means being any manual or mechanically assisted means for passing air through the connection means of the absorbent article. Desirably, the connection means is located in the periphery of the absorbent article and the discharge of the valving means is desirably directed into the absorbent article so air is forced into the product in the region between the skin and the product, which is generally remote from a fresh air source.

Additionally, the valving means of the absorbent article and/or the pumping means, if present, may contain a desiccant on the inside portions thereof. The addition of a desiccant in the valving and/or pumping means may act to further reduce the relative humidity of the air passing into the absorbent article, thereby allowing for the removal of more moisture from the article as the air having a reduced humidity is passed into or through the article.

### BRIEF DESCRIPTION OF THE DRAWINGS

FIG. 1 shows a one way valve.

FIGS. 2A and 2B show a one way valving mechanism using two pliant sheets in a cross-sectional view.

FIG. 3 shows a bellows with a resilient component within.

FIG. 4 shows an outside view of one example of a diaper with a pumping means incorporated.

FIG. 5 shows a view of the inside of the diaper of FIG. 4.

### DEFINITIONS

As used herein, the term “air” is intended to include any gas or gaseous combination (e.g. atmospheric air, oxygen or the like) which may be passed through tubing and which may come into contact with the human body.

As used herein, the term “biconstituent fibers” refers to fibers which have been formed from at least two polymers extruded from the same extruder as a blend. The term “blend” is defined below. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein the term “blend” means a mixture of two or more polymers while the term “alloy” means a sub-class of blends wherein the components are immiscible but have been compatibilized. “Miscibility” and “immiscibility” are defined as blends having negative and positive values, respectively, for the free energy of mixing. Further, “compatibilization” is defined as the process of modifying the interfacial properties of an immiscible polymer blend in order to make an alloy.

As used herein and in the claims, the term “comprising” is inclusive or open-ended and does not exclude additional unrecited elements, compositional components, or method steps.

As used herein, the term “conjugate fibers” refers to fibers which have been formed from at least two polymers extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75/25, 50/50, 25/75 or any other desired ratios. The fibers may also have shapes such as those described in U.S. Pat. Nos. 5,277,976 to Hogle et al., and 5,069,970 and 5,057,368 to Largman et al., hereby incorporated by reference in their entirety, which describe fibers with unconventional shapes.

As used herein, the term “disposable” includes being disposed of after a single use and not intended to be washed and reused.

“Front” and “back” are used throughout this description to designate relationships relative to the garment itself, rather than to suggest any position the garment assumes when it is positioned on a wearer.

As used herein, the terms “inward” and “outward” refer to positions relative to the center of an absorbent garment, and particularly transversely and/or longitudinally closer to or away from the longitudinal and transverse center of the absorbent garment.

“Feminine hygiene products” means sanitary napkins or pads, tampons and panty-liners.

As used herein, the term “liquid” means a non-particulate substance and/or material that flows and can assume the interior shape of a container into which it is poured or placed.

As used herein, the term “liquid communication” means that liquid is able to travel from one layer to another layer, or one location to another within a layer.

As used herein, the terms “longitudinal” and “transverse” have their customary meanings. The longitudinal axis lies in the plane of the article when laid flat and fully extended and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “target area” refers to the area or position on a personal care product where an insult is normally delivered by a wearer.

### Test Methods

Material Caliper (Thickness):

The caliper of a material is a measure of thickness and is measured at 0.05 psi with a Starret-type bulk tester, in units of millimeters.

Density:

The density of the materials is calculated by dividing the weight per unit area of a sample in grams per square meter (gsm) by the bulk of the sample in millimeters (mm) at 68.9 Pascals and multiplying the result by 0.001 to convert the value to grams per cubic centimeter (g/cc). A total of three samples would be evaluated and averaged for the density values.

### DETAILED DESCRIPTION OF THE INVENTION

Personal care absorbent articles include such items as diapers, training pants, swimwear, feminine hygiene products such as sanitary napkins, panty-liners and tampons, incontinence garments and devices, wound dressings, bandages, absorbent pads and the like. The most basic design of all such articles typically includes a bodyside liner, an outercover and an absorbent core disposed between the bodyside liner and the outercover.

The lack of air flow into personal care products has been a long standing concern because of adverse skin effects which are believed to be promoted by continuous exposure to moisture, high temperatures and the enzymes and other substances in bodily fluids. Many attempts to correct these adverse effects have been made, including the provision of materials designed to wick and hold liquids away from the skin, or to increase the breathability or air permeability of the personal care product components. Breathable diaper outercovers, for example, have been developed to allow increased air exchange and flow into the product and so improve the environment adjacent the skin.

In light of the low air flow within personal care products, it is an object of this invention to provide a dynamically breathable personal care product wherein air is drawn from or forced into the product in the region between the skin and the product. Dynamic breathability, i.e., the forcing of air into and/or the evacuation of air from a product, should not be confused with the provision of breathable materials which merely allow air to pass through them in a passive fashion.

A personal care product, for example an incontinence garment, typically has a bodyside layer, optionally a fluid transfer layer, a fluid retention layer and a garment side layer. It may also have a distribution layer or other optional layers to provide specialized functions.

The bodyside layer is sometimes referred to as a bodyside liner or topsheet. In the thickness direction of the article, the liner material is the layer against the wearer's skin and so the first layer in contact with liquid or other exudate from the wearer. The liner further serves to isolate the wearer's skin from the liquids held in an absorbent structure and should be compliant, soft feeling and non-irritating.

The bodyside liner can be surface treated with a selected amount of surfactant, such as at least about 0.1 wt % Ahcovel surfactant with respect to the weight of the liner, or otherwise processed to impart the desired level of wettability and hydrophilicity. If a surfactant is used, it can be an internal additive or applied to the layer by any conventional means, such as spraying, coating and the like, prior to the deposition of the next layer. Desirably the surfactant will present in an amount from about 0.2 wt % to 0.8 wt %.

The fluid retention layer should absorb liquid from the adjacent bodyside layer in a controlled manner such that liquid may be stored away from contact with the body. Retention materials generally comprise binder, synthetic fibers and natural fibers. While any of the layers of a personal care product may optionally contain a superabsorbent, the fluid retention layer is the most logical layer to contain such a component.

The garment side liner layer, also referred to as a backsheet or outer cover is the farthest layer from the wearer. The outer cover functions to prevent body exudates contained in an absorbent structure from wetting or soiling the wearer's clothing, bedding, or other materials contacting the personal care product. The outer cover has traditionally been formed of a thin thermoplastic film, such as polyethylene film, which is substantially impermeable to liquid but may optionally be composed of a vapor or gas permeable, microporous “breathable” material, that is permeable to vapors or gas yet substantially impermeable to liquid.

The optional fluid transfer layer, also referred to as a surge layer, is most typically interposed between and in intimate, liquid communicating contact with the bodyside liner and another layer such as a fluid distribution or retention layer. The fluid transfer or surge layer allows fluid movement through itself generally in the Z-direction, i.e. away from the bodyside and towards the garment side.

An optional distribution layer may be interposed above (toward a wearer) the fluid retention layer and should be capable of moving fluid from the point of initial deposition to where storage is desired. The fluid distribution layer, therefore, in addition to allowing fluid movement through itself in the Z-direction, also moves fluid in the X and Y directions. Distribution should take place at an acceptable rate such that the target insult area, generally the crotch area, is ready for the next insult. The time between insults can range from just a few minutes to hours, generally depending on the age of the wearer and the personal care product in question.

The breathable outercover discussed above is an example of the approach which has been used previously to increase the ability of air to enter the product. The breathable outercover allows air through, and, in bench testing, can provide good air flow rates as measured by conventional ASTM permeability testing. While a breathable outercover will permit air to flow through, in actual use there is no way to guarantee the flow of air through the product and all the way to the skin, since this method is entirely passive. The dynamic air flow provision remedies this by avoiding the exclusive reliance on passive air entry and forcing or drawing air into the product into the region between the skin and the product.

Another aspect of the invention is a valving means which comprises at least one one-way valve and a connection means. The connection means allows for the connection of a pump or pumping means and is desirably located in the periphery of the product. One location for the connection means is in the waist area, preferably as high on the product as possible, in, for example, a diaper, training pant or incontinence garment. Another suitable location for the connection means is in the leg area of, for example, a sanitary napkin, diaper, training pant or incontinence garment. Finally, in an absorbent pad, such as those which may be used on a bed or gurney, in a hospital or other care facility, the connection means may located in any portion of the periphery of the product, but is desirably located in a position readily accessible to the wearer or third party responsible for attaching or detaching the pump or pumping means from the absorbent article. Although the connection means may be located anywhere on the absorbent article it is desirable that the connection means be in a location which is readily accessible when the product is in use, that will not significantly hinder or adversely affect the wearer's range of motion or activities, and which will not be uncomfortable to the wearer or user of the product when the pump or pumping means is attached thereto. It is also desirable depending on the product involved that the location of connection means be such that it allows for the discrete attachment of a pump or pumping means.

The pump or pumping means, which need not be part of the article, may comprise any manual or mechanically assisted means for passing air through the connection means of the absorbent article. Any manual or mechanically assisted means for passing air through the connection means of the absorbent article may include, but is not limited to, any conventional pump or pumping means, a bellows, a vacuum means or other suction or evacuation device. The pump or pumping means desirably is self contained, but may also include tubing or piping attached to an air supply or suctioning device such as the ports found in a patient's room in a hospital or a cylinder of air, such as an oxygen tank or the like, which may be stationary or portable. For example, it is envisioned that a wearer of the product may need an supplemental air supply (e.g. supplemental oxygen), due to a medical condition or procedure, and that a split or a “Y” could be inserted into the tube connecting the air supply to the wearer's breathing device and that a portion of the air could be directed to the connection means of the absorbent article via a second tube connected to the “Y” in the supply tube.

It is contemplated that the pumping means may be activated by the wearer or by a third party. Desirably the pumping means is also remotely or programably operated. It is contemplated that the pumping means may include a conventional pump (manual or automated), the scale of which could vary from a finger operable pump or hand pump up to a large mechanically driven compressor or pumping means. Furthermore, it is desirable for the valving means of the absorbent article and/or the pumping means, if present, to contain a desiccant on the inside portions thereof. The addition of a desiccant in the valving and/or pumping means would act to further reduce the relative humidity of the air passing into the absorbent article, thereby allowing for the removal of more moisture from the article as the air having a reduced humidity is passed into or through the article.

It is recognized that each of the different types of absorbent articles may necessitate different amounts of air to be forced into or drawn therefrom in order to achieve the desired reduction in liquid and vapor within the article. The desired air flow rate would, of course, also be dependent on the location of the absorbent article on the body and the use thereof. Desirably the pump or pumping means (which is to be operably connected to the absorbent article) is capable of adjustment so as to enable the operator to vary the air flow rate through the connection means. An alternative embodiment would provide for the air flow rate which passes through the connection means to be adjusted at the connection means. Again, any flow rate which will allow the desired result to be achieved may be used, provided the pumping means selected can achieve such a rate, however, a flow rate in the range of about 10-500 cc/min is generally effective and desired for many of the embodiments of the present invention.

Desirably this invention directs the air passed through the connection means into the rest of the personal care product and across the skin of the wearer. It should be noted that even a low flow rate of air passing through the connection means and inherently the at least one valve can greatly increase the cumulative air flow through a product since personal care products may be worn for periods of time up to four hours and longer in some cases. The air driven into the product eventually escapes, of course, either, in the case of a incontinent garment for example, through the leg or torso openings, or through the outercover if it is permeable. As indicated above, another embodiment of the present invention evacuates the air from the product and expels the evacuated air into the environment. It is contemplated that the same style pumping means could be used to evacuate the air from the product as to pass air into the product provided that the pumping means is capable of providing a vacuum or suction means as opposed to forcing or pushing air. A device which evacuates the air from the article may reduce leakage concerns where the flow of air around the leg or torso openings is in an inward direction and the possibility of fluid being forced out through the leg or torso openings is reduced.

In one embodiment, particularly suitable for use with diapers and/or incontinent products, the pumping means comprises a bellows which includes an air containment sack or bag. The bellows may be attached directly to the connection means of the product or the bellows may further comprise piping or tubing to permit the connection of the bellows to the connection means and the article.

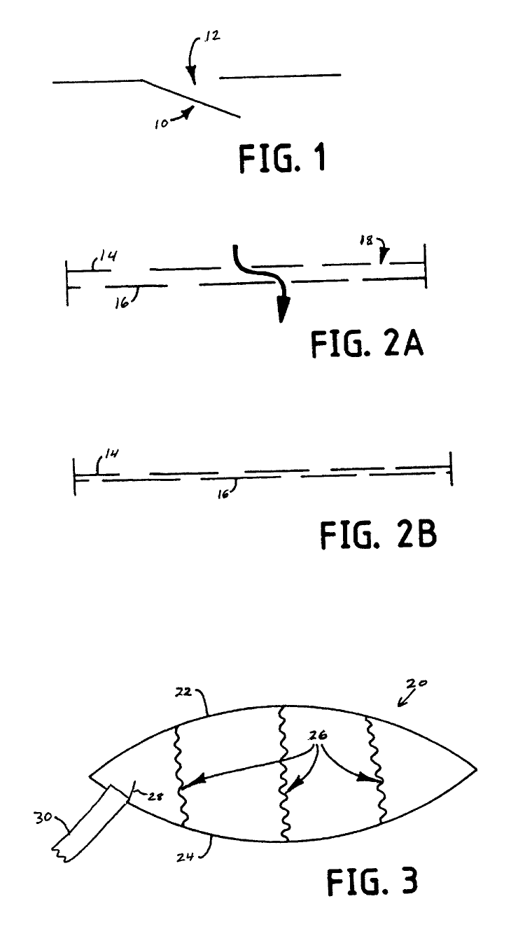

The air bag may be made out of any suitable air impermeable material. Such materials include polyolefin films and coated nonwoven fabrics. A bag made from polyethylene film would be quite suitable and inexpensive for this application. A nonwoven fabric, for example, a fabric made by the meltblowing process, and coated with a latex layer would also be suitable. Latex would be a good coating candidate because of its inherent flexibility. Extrusion coated nonwovens, such as olefin (including waxes) films also provide a cost effective alternative. In view of the fact that the air bag of the bellows is generally air impermeable, the bellows should comprise a valving means having at least two one-way valves, one to allow air to enter the bag and a second through which the air will exit. The valving means of the bellows desirably allows air into the bag on expansion and directs it into the product upon compression. In several embodiments of the present invention, it is desirable that air be drawn into the bag from outside the product. Desirably the bag includes at least two one way valves: 1) intake air from outside and 2) discharge air into the product. Any suitable one way valve would function in this service and a suitable valve is shown in FIG. 1. In FIG. 1, a pliant flap 10 will be forced to move away from the opening 12 when air flows toward the flap 10 from outside as illustrated by the arrow. If air flow is reversed, the air would force the pliant flap 10 towards opening 12 and close the opening 12. Note that the closing of the opening 12 need not completely seal the bag completely air-tight, as some air may be lost through the same valve it entered. FIGS. 2A and 2B show another valving mechanism wherein two pliant sheets 14, 16 with openings 18 which do not overlap form the valve. Air flow is again shown by the large arrow. FIG. 2A shows the pliant sheets in the open configuration and FIG. 2B shows them in the closed position.

Desirably the bellows contains a resilient component which is constructed of a flexible material which will have sufficient resiliency or compression resistance to spring back into shape after being compressed by the manual or mechanical means. The resilient component of the bellows may be elastic. One suitable material for the resilient component of the bellows is, for example, a corrugated bonded carded web of fibers. Such a material may be between 1 and 4 osy (34-135 gsm) in basis weight and have a density between about 0.01 to 0.03 gm/cc. Fibers for such a material may have a denier between about 3 and 60 dpf, desirably between about 6 and 30 dpf, and be made from thermoplastics such as polyesters, polyolefins, nylons and the like. Conjugate and biconstituent fibers are also suitable.

The shape and size of the bellows will vary based on the size of the wearer and product and the particular personal care product into which it is incorporated. FIG. 3 shows a bellows 20 having outer surfaces 22, 24 which contain a resilient component 26 between them. Each of the outer surfaces 22, 24 incorporates a one way valving arrangement. Although a variety of configurations are possible, the intake valve (not shown) is located on outer surface 22, and outlet valve 28 is shown as being on outer surface 24. Optional tubing 30 is also shown in FIG. 3. The tubing 30 should extend from the bellows 20 to the connection means (not shown) of the product.

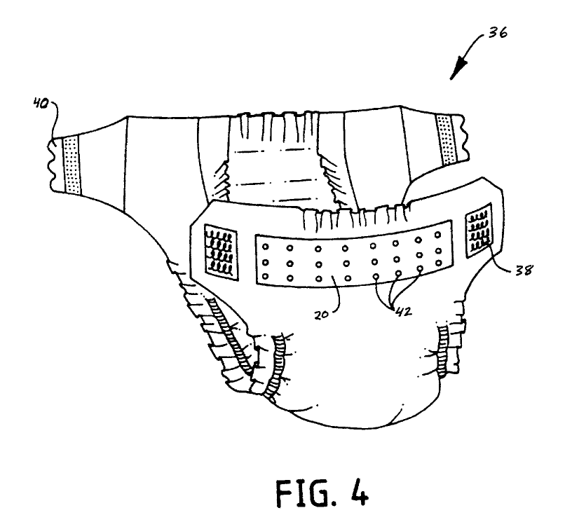

As discussed above, it is contemplated that the optional pump or pumping means may take a variety of shapes and sizes depending on the size, type and shape of the product it is used with. For example, if the pumping means were being used in the waist location of a garment such as a diaper or incontinence product, the pump could be rectangular, oblong or oval and arranged so that the long dimension is across the belly of the wearer while wearing the garment, as illustrated in FIG. 4. The discharge of the pumping means would pass through the connection means into the product and can be passed directly into the waist area of the wearer, or, to increase the amount of air going deeper into the product, can be directed by some distribution means, e.g. tubes or piping, to the crotch, back, or other area. Such distribution means need not be rigid and may collapse when air is not flowing therethrough. Further, multiple outlets may be incorporated into the valving means and/or distribution tubing which conveys air passing through the connection means into the product, so that air may be distributed in a particularly desired pattern within the product.

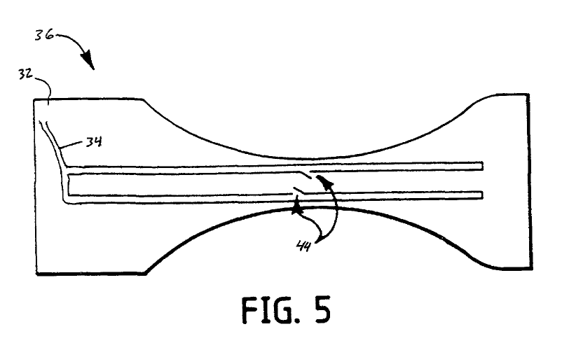

FIG. 4 shows an embodiment of the present invention with a self-contained external bellows attached to the product. FIG. 4 shows an outside view of one example of a diaper 36 with a bellows 20 attached thereto. The bellows 20 is located in the waist area on the exterior of the diaper 36 between the attachment areas 38 for the “ears” 40 of the diaper 36. The intake valving 42 of the bellows 20 is located on the front of the diaper 36. FIG. 5 shows a view of the inside of a diaper showing the discharge valving 44 which is located on the inside (toward the wearer) of the product. Connection means 32 and valving or distribution means 34 are also shown in FIG. 5.

In use, in the case of a diaper or incontinence garment for example, the waist bellows deflates when the wearer or a third party compresses the bellows. Since the intake valve 24 will close as the bellows is compressed, the deflating bag pushes its air out of the second one way (discharge) valve 28 and into the garment. As pressure on the bag of the bellows is released, the bag will begin to inflate pulling air into a first one way (intake) valve 24 and the bag from the outside. It is envisioned that a mechanically assisted pumping means may also be used. Desirably a small battery operated pump unit would be used, the pump unit would have an intake valve and an outlet valve. The unit may be programmed by the wearer, a third party or may be pre-programmed such that at the desired interval, the unit would cause air to enter the system via the unit intake valve, pass through the outlet valve of the unit, through optional tubing if necessary, and through the connection means of the absorbent article and into the desired regions of the article. One skilled in the art would recognize the pump unit could be programmed so that a specific flow rate of air is passed into the absorbent article. One skilled in the art would also recognize that any other commercially available pump or pumping means which would accomplish the desired task of passing air into the article could be used. Smaller units could be attached to the personal care product by any suitable attachment means such as a hook and loop means or to the wearer's clothing by way of a clip or the like. It is contemplated that pumps which are similar in size and shape to insulin pumps, which some diabetics must use, could be used and desirably would be of minimal hindrance to the wearer. One skilled in the art would recognize that while a smaller unit would be desired so as not to restrict the wearer's motion or otherwise hinder the wearer's movement, larger, less portable or non-portable units could also be used. Such less portable units (e.g. large air tanks, compressors, or permanent air supplies or vacuum lines) are more likely to be used in a hospital setting or the like. As indicated above, other alternative pumping means, such as air lines commonly found in hospitals or similar care facilities, would also be suitable.

Other alternatives include a pump or pumping means positioned in the leg location of a personal care product. For example, the pump or pumping means could be tubular in shape and arranged to run along the leg opening of a product, and could be mounted to the article or to the wearer's clothing. This pump or pumping means could intake air through one way valves at either or both ends and discharge it through second one way valves into the central area of the product, and be activated by, manual, remote or mechanical means. The pump or pumping means would operate in substantially the same manner as a pump or pumping means located elsewhere, however, a pump or pumping means located in the leg region of a garment type product may afford discreteness not otherwise offered by other pump or pumping means locations.

It is contemplated that the optional pump or pumping means may be permanently fixed to the absorbent article and thus disposable, or that all or part of the pump or pumping means may be reused or used with or in connection with multiple products.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means plus function claims are intended to cover the structures described herein as performing the recited function and not only structural equivalents but also equivalent structures. Thus although a nail and a screw may not be structural equivalents in that a nail employs a cylindrical surface to secure wooden parts together, whereas a screw employs a helical surface, in the environment of fastening wooden parts, a nail and a screw may be equivalent structures.

It should further be noted that any patents, applications or publications referred to herein are incorporated by reference in their entirety.

What is claimed is:

1. An absorbent article comprising a topsheet, a backsheet, and a valving means, said valving means comprising at least one one-way valve and a connection means which permits the connection of a pumping means, wherein the valving means allows air to be distributed into one or more regions of the article.

2. The absorbent article of claim 1, wherein the valving means allows air to be discharged into the article in a region between a wearer's skin and the product.

3. The absorbent article of claim 1, wherein the connection means comprises a second one-way valve.

4. The absorbent article of claim 1, wherein said valving means comprises a first one way valve which intakes air from outside said product and a second one way valve which discharges air into said product.

5. The absorbent article of claim 1, wherein the valving means further comprises piping or tubing which operably connects the connection means and the at least one one-way valve.

6. The absorbent article of claim 1, wherein the pumping means comprises any manual or mechanically assisted means for passing air through the connection means of the absorbent article.

7. The absorbent article of claim 6, wherein the pumping means comprises an external bellows.

8. The absorbent article of claim 7, wherein the pumping means further includes a desiccant placed inside of the external bellows.

9. The absorbent article of claim 7, wherein the pumping means further comprises piping or tubing to permit the connection of the pumping means to the article.

10. The absorbent article of claim 9, wherein the bellows means comprises an air bag made from materials selected from the group consisting of films and coated nonwoven fabrics.

11. The absorbent article of claim 10, wherein said air bag is extrusion coated nonwoven.

12. The absorbent article of claim 10, wherein said air bag is nonwoven fabric coated with latex.

13. The absorbent article of claim 7, wherein said bellows comprises valving means having at least two one way valves.

14. The article of claim 1 as a garment having a waist area, wherein the connection means is located in the waist area of the article.

15. The article of claim 1 as a garment having a leg area, wherein the connection means is located in the leg area of the article.

16. The absorbent article of claim 6, wherein said pumping means is permanently connected.

17. The absorbent article of claim 6, wherein said pumping means is capable of use with one or more articles.

18. The absorbent article of claim 6, wherein the external pumping means is self-contained.

19. The absorbent article of claim 6, wherein the external pumping means is attached to the article.

20. The absorbent article of claim 1 which is a personal care product.

21. The absorbent article of claim 20 which is a diaper.

22. The absorbent article of claim 20 which is a training pant.

23. The absorbent article of claim 20 which is swimwear.

24. The absorbent article of claim 20 which is an incontinence product.

25. The absorbent article of claim 20 which is a bandage.

26. The absorbent article of claim 20 which is a sanitary napkin.

27. The absorbent article of claim 20 which is an absorbent pad.

28. A personal care product comprising a topsheet, a backsheet, and a distribution means, said distribution means comprising distribution piping and a connection means which permits the connection of a pump, wherein the distribution means facilitates air exchange in the product, thereby reducing the moisture encountered by the skin of a wearer.

29. The personal care product of claim 28, wherein distribution means allows air to be discharged into one or more regions of the product.

30. The personal care product of claim 28, further comprising a pump adapted to be actuated by manual or mechanically assisted means and force air into or draw air from the absorbent article in a region between a wearer's skin and the article.

31. The personal care product of claim 30, wherein the pump may be self-contained.

32. The personal care product of claim 30, wherein the pump is readily removable from the product.

33. The personal care product of claim 28, wherein the personal care product is selected from a diaper, training pant, swimwear, absorbent underpant, adult incontinence product, feminine hygiene product, absorbent pad, wound dressing and bandage.
